(19) United States
(12) Patent Application Publication
(10) Pub. No.: US 2008/0187599 A1
(43) Pub. Date: Aug. 7, 2008

(54) Method and Apparatus for Pressure Sodding a Biological Vascular Conduit

(76) Inventors: Paul J. Dimuzio, Maple Glen, PA (US); Patrick J. Greaney, Philadelphia, PA (US); Christopher A. Dimatteo, Queen Creek, AZ (US)

Correspondence Address: DAVID S. RESNICK, NIXON PEABODY LLP, 100 SUMMER STREET, BOSTON, MA, 02110-2131, US

(21) Appl. No.: 11/885,916

(22) PCT Filed: Mar. 8, 2006
(86) PCT No.: PCT/US06/08224

§ 371 Date: Feb. 1, 2008

Related U.S. Application Data
(60) Provisional application No. 60/659,835, filed on Mar. 8, 2005.

(51) Int. Cl. A61K 35/12; C12M 3/02

(52) U.S. Cl. 424/569; 424/520; 435/304.1; 424/93.7

(57) ABSTRACT

The present invention is directed to a method for lining a biological vascular conduit with cells. The method utilizes a suitable biologic tube conduit with luminal characteristics that simulate exposed basement membrane to allow for cell attachment. The biologic conduit is secured within a seeding chamber. Cells are introduced into the conduit. Pressure is applied to the seeding chamber such that each end receives substantially equal pressure.

### BACKGROUND OF THE INVENTION

1. Field of the Invention

The present invention relates to a method and apparatus for attaching cells onto a biological vascular conduit.

2. Description of the Related Art

The ability to bypass diseased arteries remains an important technique in combating coronary and peripheral artery disease. In these circumstances, autologous tissues such as the mammary artery or greater saphenous vein are the most reliable conduits. Historically, vascular grafts have been either homografts, such as the patient's own saphenous vein or internal mammary artery, prosthetic grafts made of synthetic materials such as polyester (e.g., Dacron), expanded polytetraflouroethylene (ePTFE), and other composite materials, or fresh or fixed biological tissue grafts.

However, synthetic grafts generally have inadequate patency rates for many uses, while the harvesting of homografts requires extensive surgery which is time-consuming, costly, and traumatic to the patient. Fixed tissue grafts do not allow for infiltration and colonization by the host cells, which is essential to remodeling and tissue maintenance. Consequently, fixed tissue grafts degrade with time and will eventually malfunction.

Due to the inadequacies of these currently available synthetic and biological grafts, and the high cost and limited supply of homografts, tissue engineered grafts are being developed which are sterilized, then seeded and cultured, in vitro, with cells. These tissue engineered grafts may be superior to other grafts for use in replacement therapy in that they may display the long term dimensional stability and patency of native arteries and vessels with normal physiologic functionality.

Efforts over the past many years have focused on methods of decreasing the thrombogenicity of available vascular grafts. Much attention has been focused on cell retention of seeded/sodded cells has proven to be problematic.

### SUMMARY OF THE INVENTION

The method of the present invention involves lining a biologic vascular conduit with cells via a pressurized system; the method includes the steps of: (a) utilizing a suitable biologic tube conduit with luminal characteristics that simulate exposed basement membrane and allow for cell attachment; (b) securing the aforementioned biologic conduit within the seeding chamber; (c) introducing cells into the conduit; and (d) pressuring the apparatus via both ends of the seeding chamber, such that each end receives substantially equal pressure.

The invention discloses a unique method for sodding cells onto biologic vascular conduits, including, but not limited to, acellular collagen scaffolds and vascular tissue grafts decellularized by a variety of techniques. It should be noted that the degree of conduit porosity is of minimal concern with this particular method.

Any variety of standard cell harvesting techniques may be used to obtain cells. One such example may be found described in Jarrell et al., J Vasc. Surg. 13:733-734 (1991). Additionally, a wide variety of cells may be utilized for the procedure. Examples include, but are not restricted to: microvessel derived endothelial cells, preadipocytes, fibroblasts, mixed isolates, etc. Autologous, allogenic, and xenogenic sources may all be employed in the method of the invention.

Cells may be isolated using, for example, the method of Allen, Methods in Cell Science, 19:285-294 (1998). The references cited herein are fully incorporated herein by reference.

To sod the cells onto the biologic vascular conduit, a suspension of cells is injected via one end of the apparatus into the lumen of the conduit. The suspension is then mixed by gently drawing the suspension back and forth through each end of the conduit in order that the cell suspension reaches equilibrium. At the completion of this step, the system is pressurized via both ends such that each end receives equal pressure. Of note, the pressure may be derived from any variety of sources, including but not limited to gas, fluid, or mechanical sources. The conduit is then rotated along its axis at regular intervals to minimize gravitational effects and further ensure even removed and the sodding of the biologic vascular conduit is complete.

The apparatus according to the present invention includes a fluid reservoir, a pressure source pressure, at least one graft treatment chamber, and a tube for supporting the graft in the treatment chamber. The pressure source may include a any variety of pressure sources, including but not limited to gas, fluid, or mechanical sources.

Once the vascular graft has reached the desired level of cell density, a preservative may then be pumped into treatment chamber. Once the treatment chamber is filled with the preservative, the inlet ports and outlet ports of the chambers may be closed, again creating a sealed chamber which may then be used to store and/or ship the cultured and preserved vascular graft. Preferably, the preservative is a cryo-preservative so that the graft may be frozen in the chamber. In this manner, the sealed treatment chamber may be used to sterilize, culture, store, and ship vascular grafts or other prostheses.

The apparatus and method of the present invention preferably allow the graft to be seeded in less that one hour. Thus, if desired, the formation of the vascular graft can take place immediately before the surgical procedure.

These and other objects, features, aspects, and advantages of the present invention will become more apparent from the following detailed description of the preferred embodiment relative to the accompanied drawings, in which:

### BRIEF DESCRIPTION OF THE DRAWINGS

FIG. 1 is a perspective view of the apparatus of the present invention.

### DETAILED DESCRIPTION OF THE PREFERRED EMBODIMENTS

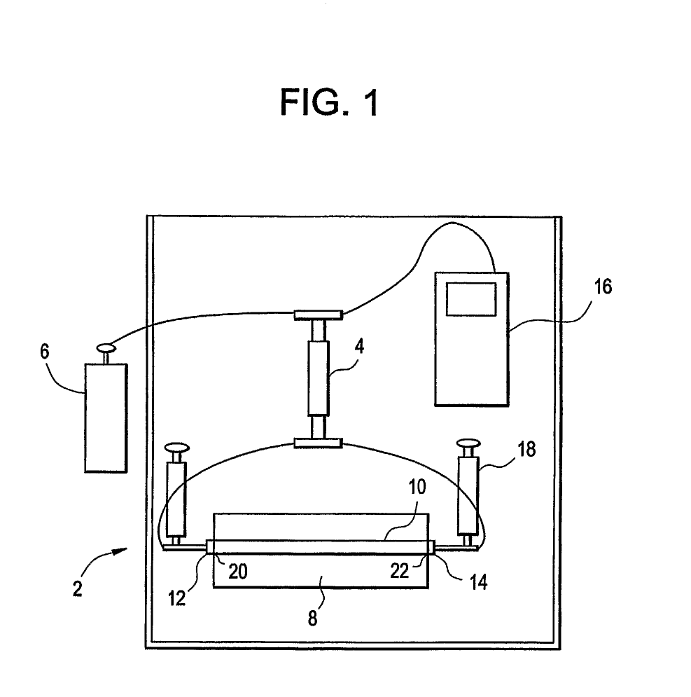

Referring to FIG. 1, an apparatus 2 for sodding a biologic vascular conduit according to the present invention is shown. Apparatus 2 includes a seeding chamber 8. In use, a biologic tube conduit 10 having luminal characteristics, which simulates an exposed basement membrane and allows for cell attachment, is secured within chamber 8 at attachment members 20 and 22 using, for example, suitable clasps (not shown). Conduit 10 is rotatably disposed within chamber 8. onto biologic tube conduit 10, a suspension of cells is injected into one port of chamber 8. A pressure source 18 is located at each port 12, 14. Each pressure source 18 can be adjusted and controlled via a fluid reservoir 4 and an air tank 6 that communicates with each pressure source 18 via conventional tubing or hose. The suspension can be mixed by alternatively opening and closing ports 12, 14 and activating respective pressure sources 18 to gently draw the suspension back and forth through each end of the conduit until the cell suspension reaches equilibrium.

It should be appreciated that pressure source 18 can take a variety of forms, including, but not limited to gas, fluid or mechanical sources. Air tank 6 communicates directly with fluid reservoir 4. By filling or depleting tank 6, the pressure within fluid reservoir 4 is adjusted, which in turn controls pressure sources 18. Tank 6 can include oxygen or any other appropriate gas or fluid. A pressure circuit measurement device 16, for example, a digital manometer, communicates with the pressure circuit.

After mixing, the conduit 10 is pressurized equally at both ends such that pressure within conduit 10 is equalized. The conduit is then rotated along its axial length by a mechanical source (not shown), for example, a motor and gearing, at regular intervals to minimize gravitational effects and further ensure the even distribution of cells. At the completion of the procedure, ports 12, 14 can be closed to remove pressure sources 18 and the sodding of the biologic vascular conduit is complete.

Once the vascular graft has reached the desired level of cell density, a preservative can be pumped into the treatment chamber through either port 12 or 14. Upon filling chamber 8 with the preservative the ports 12, 14 can again be closed to create a sealed chamber that can be used to store and/or ship the cultured and preserved vascular graft.

The apparatus and method of the present invention allow for quick seeding times, for exampl, less than one hour. This limited treatment time enables the vascular graft to be formed immediately before the surgical procedure. technique according to the present invention. This example is provided for the purpose of illustrating the invention, and should not be construed as limiting.

### EXAMPLE

### Scaffolding Preparation

Cadaveric human saphenous vein specimens were received from a tissue bank (National Disease Research Interchange, Philadelphia, Pa.). Upon arrival to the laboratory, the intact saphenous vein was dissected free from the surrounding tissue, divided into 5 cm segments, and dilated to ensure maximum surface area exposure. The specimens were rendered acellular by placing each segment into 0.075% sodium dodecyl sulfate (SDS) in a 37° C. water bath for 15 hours (Schaner, et al. J Vasc. Surg. 2004). The veins were flushed with 10 ml of phosphate buffered saline (PBS) and placed into a shaking water bath for 15 minutes. Veins were flushed an additional 5 times to remove any residual SDS. Specimens were stored in storage medium at 4° C. until use. Storage medium consisted of: M-199 (500 ml, Mediatech, Herndon, Va.), FBS (75 ml, b 12.8%, Mediatech, Herndon, Va.) HEPES (2.5 ml, 1M. Fisher Biotech, Fair Lawn, N.J.). Heparin (1 ml, Elkinssinn, inc. Cherry Hill, N.J.), Antiobiotic-Antimycotic Solution (100×) (6 ml, 10,000 U/ml Penicillin G, 25 μg/ml Amphotericin B, 10,000 μg/ml Streptomycin, Mediatech, Herndon, Va.).

### Harvest and Isolation of Preadipocytes

Human preadiopcytes were harvested fresh from patients undergoing lower extremity vascular bypass procedures at Thomas Jefferson University Hospital (Pennsylvania, Pa.). All patients consented to an elective liposuction procedure in which 30 cc of adipose tissue was obtained from the peri-umbilical region. Upon collection of the specimen, it was transported to the laboratory on ice. The specimen was filtered to remove the excess tumescent solution and washed with PBS. The adipose was incubated with collagenase 1 (40 mg/ml) for 30 minutes at 37° C. After incubation, the fat-collagenase mixture was centrifuged (1500×g for 10 minutes) and the supernatent removed. The resulting pellet was re-suspended in 10 ml of 0.1% BSA. Following the removal of all collagenase, the pellet was re-suspended in 45% Percoll gradient and centrifuged for 20 minutes at 25,000×g. The cells were then factor) and plated onto a 1% gelatinized flask. The newly isolated preadiopcytes were maintained in a constant atmosphere of 5% carbon dioxide. The culture medium was changed every 48 hours until confluence was achieved. Then the cells were split in a 1:4 ratio. Cells for experimentation were utilized at passages 3-7.

### Technique for Seeding Intact Vein Segments

Intact decellularized vein segments were secured within an in vitro bioreactor (37° C. 5% CO2) (FIG. 1) and pre-coated with 13% FBS for 1 h. Preadipocytes were introduced into the vessel lumen at lx confluence, and gas-driven intra-luminal pressure was applied from both ends of the graft over 1 h at 500 mm Hg. Real-time circuit pressure was measured via a Mannix DM8200 Digital Manometer attached to the circuit. The decellularized vein segment was rotated 90° along its axis every 15 minute. A reservoir was attached proximally to keep the circuit filled with media and allow for any porosity of the conduit. After seeding, intact segments were gently flushed with 3 cc of PBS to remove residual seeding fluid. The number of cells in this solution was measured via Coulter Counter and found to be minimal (97-98% remained).

Immediate vs. Delaved Introduction to Flow to Sodded Graft

The seeded decellularized vein segments were exposed to immediate flow×1 hr(100 cc/min) or delayed flow (20 cc/min×24 hr then 100 cc/min). Vein segments were then stained with 20 μM CellTracker Green and viewed utilizing an Olympus Fluoview inverted laser confocal microscope. Sodded segments exposed to immediate flow demonstrated excellent cell retention whereas those exposed to 24 hr “Flow-conditioning” first not only demonstrated excellent cell retention but also cell spereading and alignment with flow.

Immediate vs. Decellularized Vein Graft with Endothelial Cells

Background: Used as an arterial bypass graft, the decellularized vein is durable, has rediced antigenicity, and supports cellular repopulation in vivo. Its usefulness, however, is limited by luminal thrombogenicity secondary to endothelial loss. Herein, the method of the present invention rapidly establishes a confluent monolayer of luminal endothelial cells to address this problem. 0.075% sodium dodecyl sulfate (SDS) and divided into 4-5 cm segments. Decellularized veins were secured within an in vitro bioreactor (37° C., 5% CO2) and pre-coated with 13% fetal bovine serum for 24h. Human microvessel endothelial cells (MVEC) were introduced into the vessel lumen at lx confluence and intra-luminal pressure was applied over 1 h (0, 100, or 500 mmHg). The grafts were subjected to intra-luminal flow (100 cc/min) immediately after seeding or after a 24 h period of flow conditioning (20 cc/min). Cell attachment was measured using DNA analysis and laser confocal microscopy.

Results: MVEC attachment improved with increasing seeding pressure (0 mmHg=6.7±6.5 ng/ml, 100 mmHg=29.719.5 ng/ml, 500 mmHg=50.0±16.2 ng/ml, corresponding to <20, <60, and □95% cell attachment). Scanning microscopy confirmed increasing attachment and cell spreading on the luminal surface with increasing pressures. A >95% confluent monolayer was observed after seeding 1 h at 500 mm Hg along the circumference and length of the vein. Endothelial cells remained attached after subjecting the grafts to immediate intra-luminal flow; however, grafts that were flow-conditioned demonstrated increased cell spreading and alignment with the direction of flow.

Conclusions: Pressure sodding is more effective than gravitational force alone for resurfacing decellularized vein segments with endothelial cells in vitro. A nearly confluent monolayer of cells can be achievable within one hour. While flow conditioning was not necessary for cell attachment, it did demonstrate endothelial cell responsiveness to flow over time.

A tissue-engineered small-diameter vascular graft

As an arterial conduit, decellularized vein allograft exhibits satisfactory strength, reduced antigenicity compared to fresh allograft, and supports cellular repopulation in vivo; however, due to the lack of endothelium, it is thrombogenic. Autologous vascular cell seeding of the luminal surface overcomes this obstacle. Herein, we optimize a seeding method that efficiently establishes a confluent monolayer of cells that resists detachment under physiologic shear stress.

Methods: Decellularized human vein was seeded with vascular cells in vitro. The effect of varying seeding time, surface pre-coat, seeding density and intra-luminal pressure on cell attachment was evaluated using residual cell count, DNA quantification and laser confocal microscopy. Cell retention was measured similarly after exposure to shear stress within a pulsatile flow circuit.

Results: Under gravitation force, cell attachment occurred as early as 30 min (52±5%), neared maximum by 2 h (82±16%) and remained stable over 24 h (89±10%). Establishing a confluent monolayer required seeding with a minimum of 2× confluent number of cells. Neither serum pre-coat nor pressure (300 mmHg×1 h) enhanced attachment (P>0.05). Pressure-seeded monolayers remained intact superior to statically seeded grafts following exposure to shear stress up to 90 dyne/cm²×24 h.

Conclusions: A confluent monolayer of cells is rapidly established upon decellularized human vein without the need for serum pre-coat or pressure. Nevertheless, pressure seeding allows for cell retention even when monolayers are exposed to supra-physiologic shear stress. Future in vivo testing will determine the durability to this fully tissue-engineered vascular graft.

Although the present invention has been described in relation to particular embodiments thereof, many other variations and modifications and other uses will become apparent to those skilled in the art. It is preferred therefore, that the present invention be limited not by the specific disclosure herein, but only by the appended claims.

What is Claimed is:

1. A method for lining a biologic vascular conduit with cells comprising the steps of:
(a) utilizing a suitable biologic tube conduit with luminal characteristics that simulate exposed basement membrane to allow for cell attachment;
(b) securing the biologic conduit within a seeding chamber;
(c) introducing cells into the conduit; and
(d) pressuring the seeding chamber at both ends, such that each end receives substantially equal pressure.

2. The method of claim 1, wherein the biologic tube conduit is an acellular collagen scaffold or a decellularized vascular tissue graft.

3. The method of claim 1, further comprising rotating the conduit along its axis.

4. The method of claim 1, wherein the cells are microvessel derived endothelial cells, preadipocytes, fibroblasts, or mixed isolates.

5. The method of claim 1, wherein the cells are isolated from a subject to receive the vascular conduit.

6. The method of claim 1, further comprising adding a preservative into its chamber when the desired level of cell density was reached.

7. An apparatus for pressure sodding a biologic vascular conduit comprising:
at least one graft treatment chamber having opposed ends;
an attachment member at each end of the treatment chamber to secure a biologic tube conduit within said treatment chamber; and
an adjustable pressure source located at each end of the treatment chamber,
wherein substantially equal pressure can be applied at each end of the conduit.

8. The apparatus of claim 2, wherein the biologic tube conduit is movably disposed.
